(19) United States
(12) Patent Application Publication
(10) Pub. No.: US 2023/0124321 A1
(43) Pub. Date: Apr. 20, 2023

(54) PREDICTING PERFORMANCE OF CLINICAL TRIAL FACILITATORS USING PATIENT CLAIMS AND HISTORICAL DATA

(71) Applicant: Janssen Research & Development, LLC, Raritan, NJ (US)

(72) Inventors: Hans Roeland Geert Wim Verstraete, Shilde (BE); Francisco Xavier Talamas, Livingston, NJ (US); Nikolay Vladimirovitch Manyakov, Heverlee (BE); Geoffrey Jerome Kip, Philadelphia, PA (US)

(21) Appl. No.: 17/501,119

(22) Filed: Oct. 14, 2021

(51) Int. Cl. G16H 10/20; G16H 10/60; G06N 20/00

(52) U.S. Cl. CPC G16H 10/20; G16H 10/60; G06N 20/00

(57) ABSTRACT

A clinical trial site evaluation system applies a machine learning technique to predict recruitment performance of a candidate clinical trial facilitator (such as a clinical trial site or a clinical trial investigator) for a clinical trial based on patient claims data or other data associated with the candidate clinical trial facilitator. In a training phase, a training system trains the machine learning model based on historical recruitment data associated with historical clinical trials and patient claims data (or other data) associated with the clinical trial facilitators associated with those trials. In a prediction phase, the machine learning model is applied to claims data (or other data) associated with candidate clinical trial facilitators to predict recruitment performance.

### BACKGROUND

### Technical Field

The described embodiments relate to a machine learning technique for predicting performance of clinical trial facilitators including sites and investigators.

### Description of the Related Art

In the pharmaceutical industry, clinical trials play a key role when bringing a new treatment to market. Clinical trials are important to ensure that treatments are safe and effective. However, success of a clinical trial depends on recruiting enough eligible participants, which in turn depends on identifying specific trial sites and responsible trial investigators that are likely to produce high recruitment performance.

### BRIEF DESCRIPTION OF THE DRAWINGS

FIG. 1 is an example embodiment of a clinical trial facilitator evaluation system.

FIG. 2 is an example embodiment of a training system for training a machine learning model to predict performance of a clinical trial facilitator.

FIG. 3 is an example embodiment of a prediction system for generating performance predictions for a candidate clinical trial facilitator.

FIG. 4 is an example embodiment of a process for training a machine learning model to predict performance of a clinical trial facilitator.

FIG. 5 is an example embodiment of a process for generating performance predictions for a candidate clinical trial facilitator.

FIG. 6 is an example result of an execution of the clinical trial facilitator evaluation system.

FIG. 7 is a chart illustrating a first set of analytical data associated with predicted recruitment performance of a first candidate clinical trial facilitator based on an example execution of the clinical trial facilitator evaluation system.

FIG. 8 is a chart illustrating a second set of analytical data associated with predicted recruitment performance of a second candidate clinical trial facilitator based on an example execution of the clinical trial facilitator evaluation system.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

A clinical trial site evaluation system applies a machine learning technique to predict recruitment performance of a candidate clinical trial facilitator (such as a clinical trial site or a clinical trial investigator) for a clinical trial based on patient claims data or other data associated with the candidate clinical trial facilitator. In a training phase, a training system trains the machine learning model based on historical recruitment data associated with historical clinical trials and patient claims data (or other data) associated with the clinical trial facilitators associated with those trials. In a prediction phase, the machine learning model is applied to claims data (or other data) associated with candidate clinical trial facilitators to predict recruitment performance.

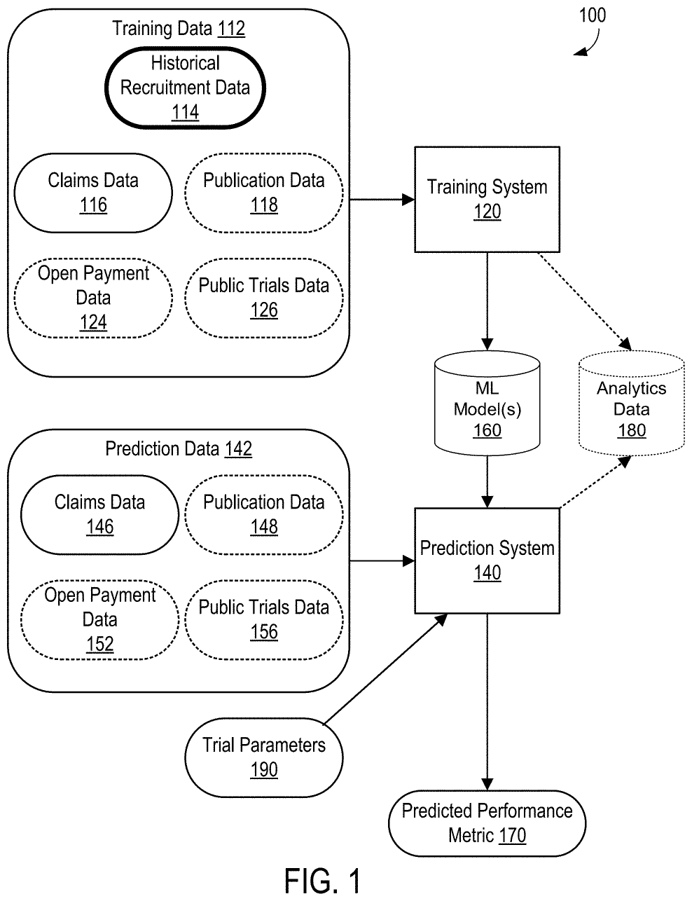

FIG. 1 illustrates an example embodiment of a clinical trial facilitator evaluation system 100 that applies a machine learning approach to predict performance of clinical trial facilitators. A clinical trial facilitator can include any human or organizational entity that participates in facilitation of the clinical trial such as a clinical trial site (e.g., a hospital, a private medical practice, a clinical research center, or other healthcare organization) or a clinical trial investigator (e.g., a doctor, a nurse, a pharmacist, a resident, an assistant, or other healthcare practitioner), or any combination thereof.

The clinical trial site evaluation system 100 comprises a training system 120 and a prediction system 140. The training system 120 trains one or more machine learning models 160 based on a set of training data 112. The prediction system 140 then applies the one or more machine learning models 160 to a set of prediction data 142 associated with one or more candidate clinical trial facilitators to generate a predicted performance metric 170 of the candidate clinical trial facilitators for a future clinical trial. The future clinical trial may be defined by a set of trial parameters 190 indicative of the purpose of the clinical trial and any specific desired outcome. For example, the trial parameters 190 may specify a specific treatment being evaluated, a timeframe for the trial, a number of participants desired, characteristics of those participants. The predicted performance metric 170 may be used to evaluate the candidate clinical trial facilitator relative to other potential candidate clinical trial facilitators. Optionally, the training system 120 and/or the predictions system 140 may furthermore output analytics data 180 that provides insight into learned relationships in the training data 112 and prediction data 142. For example, the analytics data 180 quantify the impact of different features of the training data 112 or prediction data 142 on the observed or predicted recruitment levels. This analytical data 180 may be useful together with the predicted performance metric 170 to enable an organizer to reach an informed decision in selecting a clinical trial facilitator. Furthermore, the analytical data 180 may be used to improve the training system 120 and refine the machine learning model 160.

The training data 112 includes at least a set of historical recruitment data 114 and a set of claims data 116. The training data 112 may optionally also include other types of data such as publication data 118, open payment data 120, and public trials data 122, as will be described in further detail below.

The historical recruitment data 114 is indicative of historical recruitment performance for prior clinical trials. The historical recruitment data 114 may include for example, a total number of eligible enrollees of a historical clinical trial, an enrollment rate (e.g., enrollees per specific time period) of the historical clinical trial, or other metric. The historical recruitment data 114 may directly specify one or more performance metrics or may include data from which one or more historical performance metrics can be derived. In an embodiment, the historical recruitment data 114 may include, for example, the following fields (if known/applicable) for each historical clinical trial:
Investigator Name
Facilitator ID (Recruitment) (e.g., Investigator ID (Recruitment) and/or Site ID (Recruitment))
Site Name
Location (e.g., country, state, area, city, zip code, street)
Trial ID
Site recruitment start date (or estimate)
Site recruitment closing date (or estimate)
Number of patients enrolled

The claims data 116 describes health insurance claims resulting from healthcare treatment received at a set of healthcare sites where prior historical clinical trials were implemented. The claims data 116 may describe, for example, specific treatments, procedures, diagnoses, and prescriptions for patients evaluated or treated at one of the healthcare sites where a prior historical clinical trial was implemented or by an investigator associated with the historical clinical trial. In an embodiment, the claims data 116 may include, for example, the following fields (if known/applicable) for each claim record:
Facilitator ID (Claims) (e.g., Site ID and/or Investigator ID (National, e.g., NPI))
Site Name
Location
Patient ID
Claims (e.g., date, ICD codes, procedure codes, A-V Codes, etc.)
Pharmacy data (e.g., date, dosage, NDC codes, treatment name, etc.)
Lab data
Electronic Health Records (EHR) that can be linked to a specific Facilitator ID

The publication data 118 describes publications associated with a historical clinical trial facilitator associated with a historical clinical trial. For example, a relevant publication may be one that is authored by an investigator associated with a historical clinical trial site or otherwise connected to the historical clinical trial site. In an embodiment, the publications data 118 may include, for example, the following fields (if known/applicable) for each publication:
Authors
Titles
Abstract

The open payment data 122 describes healthcare-related payments received by a site or specific investigator that took part in a historical clinical trial. In an embodiment, the open payment data may include, for example, the following fields (if known/applicable) for each payment record:
Payer
Receiver
Payment amount
Reason

The public trials data 126 describes government-published public data relating to the historical clinical trials. This data may be obtained from a public government database such as clinicaltrials.gov.

In some embodiments, the training data 112 may include other data types instead of, or in addition to, those described above. For example, the training data 112 may include data derived from Electronic Health Records (EHR), pharmacy data, lab data, or unstructured data such as notes from a health care provider.

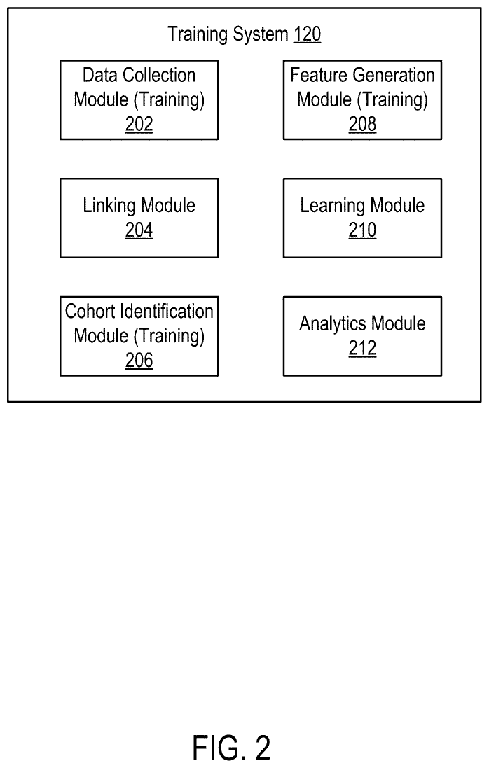

The training system 120 trains one or more machine learning models 160 based on the training data 112. Here, the one or more machine learning models 160 describes learned relationships between the historical recruitment data 114 and the claims data 116, publication data 118, open payment data 120, and/or public trial data 122. The machine learning model 160 can thus predict how features of the claims data 116, publication data 118, open payment data 120, and/or public data 122 may be indicative of different performance outcomes (e.g., in terms of total recruitment or recruitment rate) of clinical trials. The training system 120 may optionally also output analytics data 180. Here, the analytics data 180 may describe learned correlations between features of the historical recruitment data and the claims data 116, publication data 118, open payment data 120, and public trials data 122 to identify specific features highly indicative of strong recruitment performance. An example embodiment of a training system 120 is described in further detail below with respect to FIG. 2.

A prediction system 140 applies the one or more machine learning models 160 to a set of prediction data 142 to generate a predicted performance metric 170 for a planned clinical trial (as described by the trial parameters 190) facilitated by a candidate clinical trial facilitator. Here, the predicted performance metric 170 may comprise, for example, a predicted total number of eligible enrollees or a predicted enrollment rate (e.g., enrollments per relevant time period). The prediction system 140 may furthermore generate analytical data 180 indicative of the relative impacts of different features on the predicted performance metric 170.

The prediction data 142 includes claims data 146 associated with a candidate clinical trial facilitator. The set of candidate clinical trial facilitators may include those for which past historical recruitment data is not necessarily available or known. The prediction data 142 may furthermore optionally include publication data 148 and/or open payment data 154 associated with the candidate clinical trial facilitator. Furthermore, the prediction data 142 may include public trial data 156 associated with any ongoing or past trials of the candidate clinical trial facilitator. The claims data 146, publication data 148, open payment data 154, and public trial data 156 may be structured similarly to the claims data 116, publication data 118, open payment data 124, and public trial data 126 used in training data 112 described above.

The training data 112 and prediction data 142 may be stored to respective databases (or a combined database) at a single location or as a distributed database having data stored at multiple disparate locations. In an embodiment, different elements of the training data 112 and prediction data 142 may be stored to separately operated database systems accessible through respective database interfacing systems. Prior to processing, data may be imported to a common database that stores inputs, outputs, and intermediate data sets associated with the clinical trial facilitator evaluation system 100.

The training system 120 and prediction system 140 may each be implemented as a set of instructions stored to a non-transitory computer-readable storage medium executable by one or more processors to perform the functions attributed the respective systems 120, 140 described herein. The training system 120 and prediction system 140 may include distributed network-based computing systems in which functions described herein are not necessarily executed on a single physical device. For example, some implementations may utilize cloud processing and storage technologies, virtual machines, or other technologies.

FIG. 2 illustrates an example embodiment of a training system 120. The training system 120 comprises a data collection module 202, a linking module 204, a cohort identification module 206, a feature generation module 208, a learning module 210, and an analytics module 212. Alternative embodiments may comprise different or additional modules.

The data collection module 202 collects the training data 112 for processing by the training system 120. In an embodiment, the data collection module 202 may include various data retrieval components for interfacing with various database systems that source the relevant training data 112. For example, the data collection module 202 may execute a set of data queries (e.g., SQL or SQL-like queries) to obtain the relevant data.

The linking module 204 links data obtained by the data collection module 202 based on a combination of exact matching and fuzzy matching techniques. Here, exact matching may identify matches between different data sources to identify respective records associated with the same clinical trial facilitator. Fuzzy matching may be used to identify data referring to the same entity despite variations in how the identifying data is presented in the different data sources. For example, fuzzy matching may be used to identify matches between corresponding records that differ in their use full or abbreviated names, complete or incomplete data fields, or other disparities in the stored data.

In an embodiment of a multi-step linking approach, the linking module 204 first links the historical recruitment data 114 and claims data 116. Here, the linking module 204 first matches the investigator IDs in the historical recruitment data 114 to the investigator IDs in the claims data 116. A matching score is generated in which exact matches of investigator information fields (e.g., a match of name, address, country, zip code, or specialty) each result in a score of 1, while a partial match results in a score between 0 and 1. A combined score (e.g., based on a sum or average of the partial scores) expresses a likelihood that an investigator ID in the claims data 116 corresponds to an investigator ID in the historical recruitment data 114. If the likelihood exceeds a predefined threshold, the historical recruitment data and claims data 116 associated with the matched investigator are linked to a common investigator ID. Since investigator IDs are linked to site-level information in the historical recruitment data 114 and claims data 116, this site-level information can also be compared between the data records where matching investigator IDs were found. If the site-level data sufficiently matches, the site IDs can also be linked into a common site ID. In cases where an investigator ID is associated with multiple different site IDs in the historical recruitment data 114 an claims data 116, priority is given to the site IDs with a higher number of claims. Additionally, exact and fuzzy matching techniques may be performed to directly identify matches between the site IDs in the historical recruitment data 114 and the site IDs in the claims data 116 to find additional matches. The site IDs may be matched based on information fields such as facility name, address, city, zip code, and state using a similar technique as described above.

The publication data 118 and open payment data 122 may also be linked to investigator-level and/or site-level records based on exact or fuzzy matches. Here, the linking module 204 identifies matches between the investigator IDs in the previously linked data records and the author fields of the publication data 118 and/or receiver information fields of the open payment data 122. Fuzzy matching techniques like those described above may be utilized to identify corresponding entities even in the presence of variations in the specific data stored to the different systems.

As a result of the linking process, data records are created that associate, for each historical clinical trial, the historical recruitment data 114 (including recruitment performance metrics) associated with that trial to all available data relating to the site at which the historical clinical trial was performed and/or the investigator responsible for the historical clinical trial.

The cohort identification module 206 processes the claims data 116 to identify one or more patient cohort data sets pertaining to a patient cohort. Each patient cohort data set comprises a subset of the patient claim data 116 for patients in the patient cohort having a defined relevance (e.g., defined by a filtering criteria) to one or more of the historical clinical trials. The filtering criteria may be designed such that the patient cohort includes patients that would have potentially been eligible for the historical trial. For example, a patient cohort data set may include claims data 116 referencing a specific diagnosis, received treatment (e.g., drug usage, administration, or procedure), or prescription relevant to one or more specific historical clinical trials. Multiple cohort data sets for different patient cohorts may be generated for each historical clinical trial that are each based on a different set of relevant filtering criteria. Furthermore, the same patient cohort data set may be relevant to more than one different clinical trial.

In one example, a patient cohort data set for a historical clinical trial relating to a treatment for inflammatory bowel disease (IBD) may be created by filtering claims data to identify claim records having a Crohn's disease diagnosis code (e.g., code K50 for ICD-10). Another patient cohort data set for a different clinical trial may be created by filtering claims data to identify claim records having an ulcerative colitis diagnosis code (e.g., code K51 for ICD-10). Yet another cohort data set associated with either or both of the aforementioned trials may be created that includes only claim records for patients having previously taken a particular treatment associated with IBD after having been diagnosed with Crohn's disease or ulcerative colitis for the respective underlying trial.

In another example, a patient cohort data set for a historical clinical trial relating to a treatment for pulmonary arterial hypertension (PAH) may be created by filtering claims data for claims having a relevant diagnostic code (e.g., ICD10 code 127 corresponding to primary pulmonary hypertension). A second cohort data set may be identified that includes patient claims for patients treated with a PAH drug within 6 months from diagnosis. A third (narrower) patient cohort data set may be identified to include patient claims from the second cohort limited to those that also received an echocardiograph or right heart catherization.

A patient cohort data set may be relevant to multiple different historical clinical trials. For example, the third patient cohort described above for patients receiving an echocardiograph or right heart catherization may be equally relevant to other clinical trials for PAH or clinical trials for other diseases.

Cohort data sets may furthermore be time limited. In this case, the cohort identification module 206 may apply time-based filtering criteria that dictate a limited range of claims dates for inclusion in the cohort data set. The date range may be set relative to the clinical trial start date, end date, or other reference date.

The cohort identification module 206 may furthermore generate referral network data associated with the cohort data sets from referral information in the claims data 116. The referral network data is indicative of the flow of patients to and from a clinical trial facilitator. The referral network data may indicate, for example, how many patients were referred to and/or from clinical trial facilitators associated with the cohort data set, or other statistical information derived from the referral information.

The feature generation module 208 generates feature sets from the claims data 116 in each patient cohort data set and from the publications data 118, open payment data 120, and/or public trials data 122 relevant to a particular clinical trial facilitator associated with a historical clinical trial. Features sets may include features generated at the site-level (i.e., including all data relevant associated with the site), at the investigator level (i.e., including only data associated with a particular investigator), or both. Furthermore, some features may be time-limited (including only data associated with a particular time period), while other features are not necessarily time-limited.

Examples of features derived from the claims data 116 may include one or more of the following:
A count of all claims associated with a clinical trial facilitator (site and/or investigator) in the cohort data set
A count of a specific type of claim (e.g., identified by a specific claim code) associated with a clinical trial facilitator in the cohort data set (e.g., ICD10 code K50 for a cohort associated with ulcerative colitis)
A count of unique patients from a patient cohort with claims associated with a clinical trial facilitator
A count of unique patients from a patient cohort with a specific type of claim (e.g., identified by a specific claim code) associated with the clinical trial facilitator (e.g., ICD10 code K50 for a cohort associated with ulcerative colitis)
A count of unique patients from a patient cohort that had a particular procedure performed relevant for the therapeutic area or disease area associated with the clinical trial facilitator (e.g., a histopathology for bowel diseases or injection with a particular drug)
A count of unique patients from a patient cohort that received a prescription for a drug to treat a disease relating to the cohort definition associated with the clinical trial facilitator
An average number of visits per patient from a patient cohort for any claim associated with the clinical trial facilitator
An average number of visits per patient from a patient cohort for a specific type of claim (e.g., identified by a specific claim code) associated with the clinical trial facilitator (e.g., ICD10 code K50 for a cohort associated with ulcerative colitis)
A PageRank score from referral networks derived from a cohort data set that represents the connectivity level of the clinical trial facilitator
A centrality metric (e.g., using eigenvalue, degree, betweenness, harmonic . . . ) of the clinical trial facilitator in the referral network of the patient cohort
Incoming and outgoing counts of patients and visits associated with the clinical trial facilitator in the cohort data set
A count of prescriptions from the clinical trial facilitator within the cohort data set
A count of a specific procedure performed on a patient of the patient cohort associated with the clinical trial facilitator (e.g., a histopathology)

An example of a feature derived from the publication data 118 may include, for example, a count of publications by the clinical trial facilitator related to a specific disease or indication relevant to the historical clinical trial.

Examples of features derived from the open payment data 122 may include one or more of the following:
The total payments (e.g., in dollars or other currency) made to the clinical trial facilitator
The total payments made to the clinical trial facilitator that are related to research or clinical trials
The total payments made to the clinical trial facilitator associated with a specified specialty area (e.g., gastroenterology)
The total number of payment transactions received by the clinical trial facilitator
The total number of payment transactions received by the clinical trial facilitator that are related to research or clinical trials
The total number of payment transactions received by the clinical trial facilitator associated with a specified specialty area (e.g., gastroenterology)

An example of a feature derived from the public trials data 126 may include, for example, one or more counts of the ongoing trials associated with the clinical trial facilitator that are related to a specific disease or indication. Here, the counts may represent a total count of ongoing trials or may represent counts associated with treatments developed by a specific entity or set of entities.

The learning module 210 generates the machine learning model 160 according to a machine learning algorithm. The learning module 210 learns mappings between each of the feature sets described above (which each relate to a patient cohort relevant to a specific historical clinical trial) and the historical recruitment data 114 for the historical clinical trial. As described above, multiple cohort data sets and corresponding feature sets may be relevant to the same historical clinical trial and thus may each influence the training of the machine learning model 160.

The learning module 210 may generate the machine learning model 160 as a neural network, a generalized linear model, a tree-based regression model, a support vector machine (SVM), a gradient boosting regression or other regression model, or other different types of machine learning models capable of achieving the functions described herein.

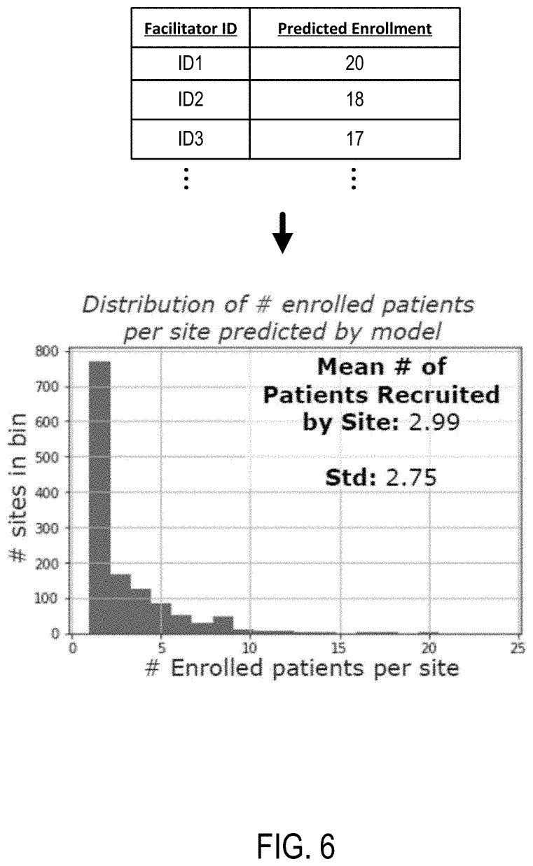

The analytics module 212 generates various analytical data associated with the machine learning model 160 and learned characteristics of the training data 112. The analytical data may be useful to illustrate the impact of different features of the training data 112 on the observed performance metrics of the historical recruitment data 114. The analytical module 212 may aggregate the analytical data into various charts, diagrams, visual representations on a map, or lists useful to present the information. For example, the analytics module 212 may output a ranked list of features that are observed to be most closely correlated with high recruitment levels. In another example, the impact associated with a particular feature may be charted over time to provide insight into the most relevant time window for predicting performance of a clinical trial site. The analytical data may be helpful to improve operation of the training system 120 and prediction system 140. For example, the analytical data may identify a limited number of features that have the highest impact to enable future training and prediction to be accomplished using a limited number of features. The analytical data may also be useful to enable researchers to make manual adjustments to operations of the training system 120 and prediction system 140 to improve performance prediction. In an embodiment, the analytics model 212 may output the analytics data as a graphical user interface that may include various charts, graphs, or other data presentations such as illustrated in FIGS. 6-8 described below.

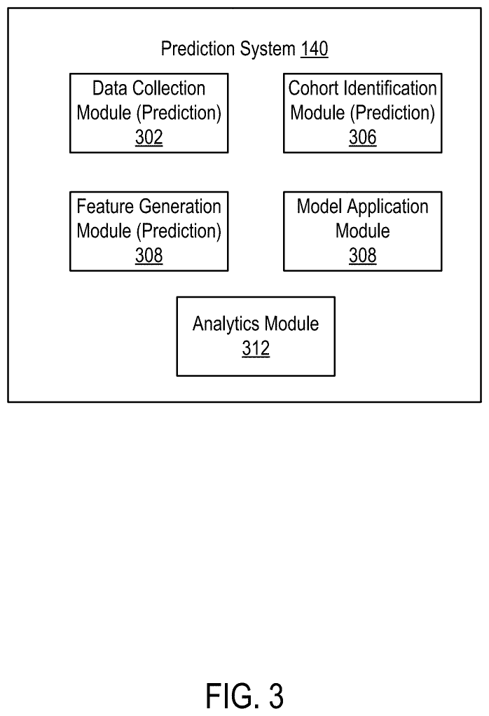

FIG. 3 illustrates an example embodiment of a prediction system 140. The prediction system 140 comprises a data collection module 302, a cohort identification module 306, a feature generation module 308, a model application module 308, and an analytics module 310. The data collection module 302, cohort identification module 306, and feature generation module 308 operate similarly to the data collection module 202, cohort identification module 206, and feature generation module 208 of the training system 120 described above but are applied to the prediction data 142 instead of the training data 112. Here, the data collection module 302 collects the claims data 146, publication data 148, open payment data 154, and public trials data 156 related to a set of candidate clinical trial facilitators (including candidate sites and/or candidate investigators) for a future clinical trial. The candidate clinical trial facilitators may lack any history of past clinical trials. The cohort identification module 306 generates one or more cohort data sets that each have some specified relevance (e.g., defined by a filtering criteria) to the future clinical trial based on the specific trial parameters 190. For consistency, the cohort identification module 306 may identify the cohort data sets in the same way (e.g., according to the same filtering criteria) as the cohort identification module 206 used in training. The feature generation module 308 derives a set of features from each cohort data set relevant to a particular candidate trial facilitator for a future clinical trial. The feature generation module 308 may generate the features according to the same techniques as the feature generation module 208 used in training. The model application module 308 then applies the machine learning model 160 to the feature set(s) derived from the feature generation module 308 (each feature set associated with a particular cohort data set) to generate the predicted performance metric 170. As described above, multiple cohort data sets and corresponding feature sets may be derived associated with the same candidate clinical trial facilitator for the same future clinical trial. In this case, the machine learning model 160 is applied to the collective feature sets to generate the predicted performance metric 170. The analytics module 312 operates similarly to the analytics module 212 described above to generate analytical data representing the relative impact of different features on the predicted performance metric 170. In an embodiment, the analytics model 312 may output the analytics data, together with the predicted performance metrics 170, as a graphical user interface that may include various charts, graphs, or other data presentations such as illustrated in FIGS. 6-8 described below.

In an embodiment, the modules 202/302, 206/306, 208/308, 212/312 are not necessarily independent and the same modules 202/302, 206/306, 208/308, 212/312 may be applied in both training and prediction. Alternatively, different instances of these modules 202/302, 206/306, 208/308, 212/312 may be used by the training system 120 and the prediction system 140.

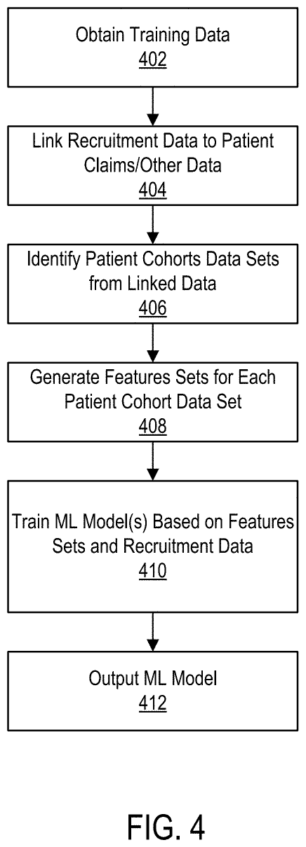

FIG. 4 is a flowchart illustrating an example embodiment of a process for training a machine learning model that can predict a performance metric 170 associated with a candidate clinical trial facilitator for a future clinical trial. The training module 120 obtains 402 training data 112 that includes historical recruitment data 114 for a set of historical clinical trials associated with a set of historical clinical trial facilitators, and historical patient claim data 116 describing historical patient claims associated with the historical clinical trial facilitators. The training module 120 may link the recruitment data 114 to the claims data 116 and any other data based on exact or fuzzy matching techniques. The training data 112 may also include publications data 118, open payment data 120, and public trials data 122 as described above. The training module 120 identifies 406 patient cohort data sets associated with the set of historical clinical trials. Each patient cohort data set comprises a subset of the historical patient claims data that relates to a corresponding historical clinical trial facilitator and that identifies a patient as meeting eligibility criteria associated with a corresponding historical clinical trial performed by the corresponding historical clinical trial facilitator. The training module 120 generates 408 respective feature sets for each of the patient cohort data sets. The training module 120 trains 410 a machine learning model 160 that maps the respective features sets for the patient cohort data sets to respective historical recruitment data 114 associated with the set of historical clinical trials. The training module 120 outputs 412 the machine learning model for application by the prediction system 140 to predict the performance of a candidate clinical trial facilitator of a future clinical trial. As described above, the training module 120 may furthermore optionally output various analytical data 180 indicative of the impact of various features of the training data 112 on the historical recruitment performance.

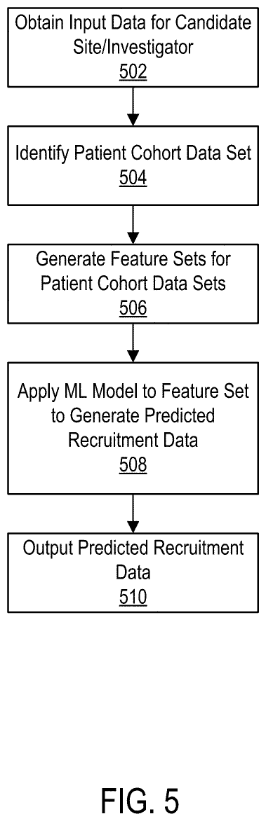

FIG. 5 is a flowchart illustrating an example embodiment of a process for predicting performance of a candidate clinical trial facilitator for conducting a clinical trial. The prediction system 140 obtains 502 input data including patient claims data 116 describing patient claims associated with a candidate clinical trial facilitator for the clinical trial. The prediction system 140 identifies 504 a patient cohort data set comprising a subset of the patient claim data that relates to a medical treatment or a condition associated with the clinical trial. The prediction system generates 506 a feature set representing the patient cohort data set. The prediction system 140 then applies 508 a machine learning model (e.g., as generated in the process of FIG. 4 above) to map the feature set to predicted recruitment data for the candidate clinical trial facilitator. The prediction system then outputs 510 the predicted recruitment data.

FIG. 6 is a graph illustrating example output data derived from an execution of the clinical trial facilitator evaluation system 100 for an example clinical trial. For this example, execution of the clinical trial facilitator evaluation system 100, the prediction system 140 outputted, for each of a plurality of candidate clinical trial sites, the total number of patients per site that were predicted to enroll in an example clinical trial. The predictions were then ranked and binned. A chart illustrates the number of sites predicted to fall into each bin (each corresponding to a specific predicted number of enrolled patients). In this example execution, the prediction data resulted in a mean of 2.99 patients per site with a standard deviation of 2.75.

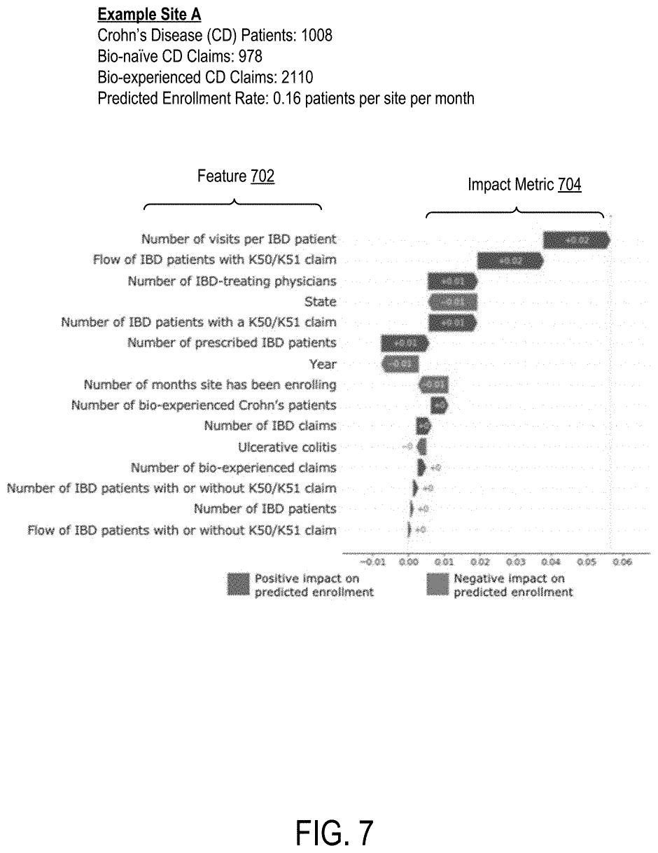

FIG. 7 is a chart illustrating a first set of analytical data derived from an example execution of the clinical trial facilitator evaluation system 100. This example related to evaluation of a candidate clinical site “A” (comprising multiple locations) for a planned clinical trial relating to a Crohn's disease (CD) treatment. The prediction system 140 ranked the candidate clinical site “A” among the top 20 sites (in terms of predicted enrollment rate) out of approximately 10,000 evaluated candidates. In this example, the training system 140 predicted an enrollment rate of 0.16 patients per month per site. The chart shows the set of impact metrics 704 calculated for various features 702. Here, the impact metric represents a contribution of the feature to a deviation from a baseline predicted enrollment rate (in this case, 0.1). Only a subset of the features is expressly shown and other features having very low impact on the results are omitted. As seen from the analytical data, the most positively impactful features were the number of visits to the site by IBD patients, the flow of IBD patients with claim codes (K50/K51) corresponding to IBD, the number of IBD patients with claims having a claim code (K50/K51) corresponding to IBD, and number of prescribed IBD patients. The most negatively impactful features included the state, year, and number of months the site has been enrolling.

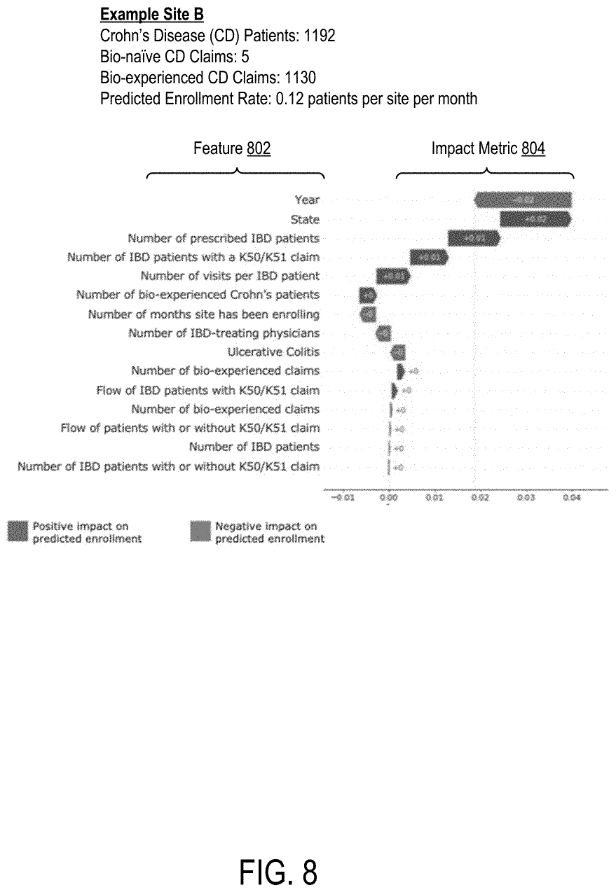

FIG. 8 is another chart illustrating a second set of analytical data derived from an example execution of the clinical trial facilitator evaluation system 100. This example related to evaluation of a candidate clinical site “B” (comprising multiple locations) for the same planned clinical trial relating to the CD treatment. The prediction system 140 also ranked the candidate clinical site “B” in the top 20 of the approximately 10000 evaluated sites, but the rank was lower than candidate clinical trial site “A”. In this example, the training system 140 predicted an enrollment rate of 0.12 patients per month per site. In this case, the most positively impactful features included its location at the state level, the number of IBD patients with a claim code (K50/K51) corresponding to IBD, the n umber of prescribed IBD patients, and the number of visits per IBD patient. The year represented the most negatively impactful feature.

Embodiments of the described clinical trial site evaluation system 100 and corresponding processes may be implemented by one or more computing systems. The one or more computing systems include at least one processor and a non-transitory computer-readable storage medium storing instructions executable by the at least one processor for carrying out the processes and functions described herein. The computing system may include distributed network-based computing systems in which functions described herein are not necessarily executed on a single physical device. For example, some implementations may utilize cloud processing and storage technologies, virtual machines, or other technologies.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A method for generating a machine learning model that predicts performance of a candidate clinical trial facilitator for conducting a future clinical trial, the method comprising:
obtaining training data including historical recruitment data for a set of historical clinical trials associated with a set of historical clinical trial facilitators, and historical patients claim data describing historical patient claims associated with the historical clinical trial facilitators;
identifying patient cohort data sets associated with the set of historical clinical trials, each patient cohort data set comprising a subset of the historical patient claims data that relates to a corresponding historical clinical trial facilitator and that identifies a patient as meeting eligibility criteria associated with a corresponding historical clinical trial performed by the corresponding historical clinical trial facilitator;
generating respective feature sets for each of the patient cohort data sets;
training the machine learning model that maps the respective respective features sets for the patient cohort data sets to respective historical recruitment data associated with the set of historical clinical trials; and
outputting the machine learning model for application by a prediction system to predict the performance of the candidate clinical trial facilitator of the future clinical trial.

2. The method of claim 1, wherein obtaining the training data further comprises:
linking the historical recruitment data with the historical patient claims data based on matching identifying information for the historical clinical trial facilitators specified in the historical recruitment data and the historical patient claims data.

3. The method of claim 1, wherein the training data further includes:
publication data describing publications associated with the historical clinical trial facilitators relating to the historical clinical trials.

4. The method of claim 1, wherein the training data further includes:
open payments data describing financial transactions associated with the historical clinical trial facilitators relating to patient care.

5. The method of claim 1, wherein the training data further includes:
public trial data describing the historical clinical trials or ongoing clinical trials associated with historical clinical trial facilitators.

6. The method of claim 1, wherein identifying the patient cohort data sets further comprises:
generating, for each of the patient cohort data sets, referral network data specifying counts of patient referrals to or from the corresponding historical clinical trial facilitator.

7. The method of claim 1, wherein generating the feature sets comprises generating at least one of the following features:
a number of the historical patient claims related to the patient cohort;
a number of ongoing clinical trials associated with the historical clinical trial facilitator;
a number of patients flowing into or out of the historical clinical trial facilitator; and
a number of the historical patient claims relating to a relevant treatment or diagnosis.

8. The method of claim 1, further comprising:
generating, based on the machine learning model, a set of impact scores indicating relative impact of different ones of the feature sets on the respective historical recruitment data; and
outputting the set of impact scores.

9. The method of claim 1, wherein training the machine learning model comprises:
applying at least one of a linear model training algorithm, an artificial neural network training algorithm, a tree-based regression algorithm, a support vector machine training algorithm, and a gradient boosting regression algorithm.

10. The method of claim 1, wherein the set of historical clinical trial facilitators comprises at least one of a clinical trial site or a clinical trial investigator.

11. A method for predicting performance of a candidate clinical trial facilitator for conducting a clinical trial, the method comprising:
obtaining input data including patient claims data describing patient claims associated with the candidate clinical trial facilitator for the clinical trial;
identifying a patient cohort data set comprising a subset of the patient claim data that relates to a medical treatment or a condition associated with the clinical trial;
determining a feature set representing the patient cohort data set;
applying a machine learning model to map the feature set to predicted recruitment data for the candidate clinical trial facilitator, the machine learning model trained based on a set of training data including historical patient claims data and historical recruitment data for a set of historical candidate clinical trial facilitators associated with a set of historical clinical trials; and
outputting the predicted recruitment data.

12. The method of claim 11, wherein the input data further includes:
publication data describing publications associated with the candidate clinical trial facilitator.

13. The method of claim 11, wherein the input data further includes:
open payments data describing financial transactions relating to patient care associated with the candidate clinical trial facilitator.

14. The method of claim 11, wherein the input data further includes:
public trial data describing historical or ongoing clinical trials associated with the clinical trial facilitator.

15. The method of claim 11, wherein identifying the patient cohort data set further comprises:
generating referral network data specifying counts of patient referrals to or from the clinical trial facilitator.

16. The method of claim 11, further comprising:
generating, based on the machine learning model, a set of impact scores indicating relative impact of different ones of the feature sets on the predicted recruitment data; and
outputting the set of impact scores.

17. The method of claim 11, wherein training the machine learning model comprises:
applying at least one of a linear model training algorithm, an artificial neural network training algorithm, a tree-based regression algorithm, a support vector machine training algorithm, and a gradient boosting regression algorithm.

18. The method of claim 11, wherein the set of candidate clinical trial facilitators comprises at least one of a clinical trial site or a clinical trial investigator.

19. A non-transitory computer-readable storage medium storing instructions for generating a machine learning model that predicts performance of a candidate clinical trial facilitator for conducting a future clinical trial, the instructions when executed by one or more processors causing the one or more processors to perform steps including:
obtaining training data including historical recruitment data for a set of historical clinical trials associated with a set of historical clinical trial facilitators, and historical patients claim data describing historical patient claims associated with the historical clinical trial sites or the historical clinical trial investigators;
identifying patient cohort data sets associated with the set of historical clinical trials, each patient cohort data set comprising a subset of the historical patient claims data that relates to a corresponding historical clinical trial facilitator and that identifies a patient as meeting eligibility criteria associated with a corresponding historical clinical trial performed by the corresponding historical clinical trial facilitator;
generating respective feature sets for each of the patient cohort data sets;
training the machine learning model that maps the respective features sets for the patient cohort data sets to respective historical recruitment data associated with the set of historical clinical trials; and
outputting the machine learning model for application by a prediction system to predict the performance of the candidate clinical trial facilitator of the future clinical trial.

20. A non-transitory computer-readable storage medium storing instructions for predicting performance of a candidate clinical trial facilitator for conducting a clinical trial, the instructions when executed by one or more processors causing the one or more processors to perform steps comprising:
obtaining input data including patient claims data describing patient claims associated with the candidate clinical trial facilitator for the clinical trial;
identifying a patient cohort data set comprising a subset of the patient claim data that relates to a medical treatment or a condition associated with the clinical trial;
determining a feature set representing the patient cohort data set;
applying a machine learning model to map the feature set to predicted recruitment data for the candidate clinical trial facilitator, the machine learning model trained based on a set of training data including historical patient claims data and historical recruitment data for a set of historical candidate clinical trial facilitators associated with a set of historical clinical trials; and
outputting the predicted recruitment data.
